(19) United States
(12) Patent Application Publication
(10) Pub. No.: US 2004/0009902 A1
(43) Pub. Date: Jan. 15, 2004

(54) CTP extended erythropoietin

(76) Inventors: Irving Boime, St. Louis, MO (US); Faud Fares, Haifa (IL)

Correspondence Address: MORRISON & FOERSTER LLP, 3811 VALLEY CENTRE DRIVE, SUITE 500, SAN DIEGO, CA, 92130-2332, US

(21) Appl. No.: 10/438,277

(22) Filed: May 13, 2003

Related U.S. Application Data
(60) Provisional application No. 60/380,506, filed on May 13, 2002.

(51) Int. Cl. A61K 38/22; C07K 14/575

(52) U.S. Cl. 514/8; 514/12; 530/398

(57) ABSTRACT

Erythropoietin containing a CTP extension and secreted from CHO cells exhibits a favorably extended biological half-life.

This application claims priority from provisional application No. 60/380,506 filed May 13, 2002. The contents of this application are incorporated herein by reference.

### TECHNICAL FIELD

The invention is directed to an improved form of erythropoietin.

### BACKGROUND ART

Erythropoietin is a naturally occurring protein which stimulates the production of red blood cells. Human erythropoietin contains 165 amino acids and the gene encoding the human protein was recovered and formed the basis for one of the first successful recombinantly produced products. The structure of erythropoietin and the gene encoding it are described in a U.S. patent awarded to Amgen, U.S. Pat. No. 4,703,008. Additional patents which describe and claim the recombinant production of this protein include U.S. Pat. Nos. 5,547,933; 5,618,698; 5,621,080; 5,756,349; and 5,955,422. The complete structure of the human erythropoietin coding sequence and means for production of the protein are described in these patents.

Attempts have been made to enhance the biological half-life of the 165 amino acid human erythropoietin protein. In one approach, the amino acid sequence has been modified to provide sites for additional glycosylation. The resulting, more highly glycosylated forms, appear to exhibit this desirable property. Isoforms of erythropoietin having specified numbers of sialic acids associated with the protein are described in U.S. Pat. No. 5,856,298. Another approach involves linking two erythropoietin moieties together as described in U.S. Pat. No. 5,747,446.

An additional method of enhancing biological half-life of proteins in general is described in U.S. Pat. No. 5,712,122. In the approach described and claimed in this patent, protein or peptide pharmaceuticals are coupled at the C-terminus to the carboxy terminal portion (CTP) of the β subunit of human chorionic gonadotropin. Presumably because additional glycosylation sites are thereby appended to the peptide, its biological half-life can be enhanced. The focus of the disclosure in the '122 patent is on the glycosylated hormones involved in reproduction and thyroid production-FSH, LH and TSH, although it is clearly recognized and claimed that proteins in general would benefit from this modification. Specifically mentioned are various growth factors, urokinase, thrombin, and interleukins. Erythropoietin is specifically mentioned but no detailed instructions for construction of CTP-extended erythropoietin are provided.

PCT publication WO 02/48194 purports to describe a form of human erythropoietin coupled to a CTP at its carboxy terminus. The fusion protein is said to have extended half-life when injected into mice.

### DISCLOSURE OF THE INVENTION

Applicants now describe the construction of a specific form of CTP-extended erythropoietin and its production in CHO cells.

### BRIEF DESCRIPTION OF THE DRAWINGS

FIGS. 1A and 1B show the results of Western blots of secreted EPO-CTP from CHO cells.

### MODES OF CARRYING OUT THE INVENTION

The specific CTP-extended erythropoietin was constructed as follows: The hEPO-CTP was constructed using overlapping PCR mutagenesis as described by Ho, S. N., et al., Gene (1989) 77:51-59. The nucleotide sequence encoding the CTP was ligated in frame at the 3′ end of the hEPO cDNA as shown below.

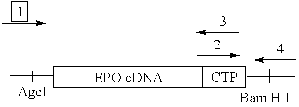

The following primers were used:

| | |
|---|---|
| Primer 1: | 5′-ACC AGA TCT ACC GGT CAT CAT GGG-3′ |
| Primer 2: | 5′-ACC TCC AGA GTG CGG ATG GAG AAG-3′ |
| Primer 3: | 5′-GAG GAG AGG GGA GAG ATG GTG TTG GTG AAA GGG -3′ |
| Primer 4: | 5′-GGG TTT GAG GAA GAG GAT GTG TGG GGT GTG GTG -3′ |

For construction of hEPO-CTP, the expression vectors, pM² hCGβ and pTG-EPO were used as a template DNA for PCR. pM² hCGβ contains the coding sequence of human hCGβ inserted into the vector pM² which is described in Matzuk, M. M et al. Proc. Natl. Acad. Sci. USA (1987) 84:6354-6358; Matzuk, M. M et al. J. Cell Biol. (1988)106:1049-1059. pTG-EPO contains the coding sequence for erythropoietin inserted into commercially available vector pTG 123 available from Invitrogen, San Diego, Calif.

In the first PCR reaction, pTG-EPO vector and primers 1 and 3 were used to generate a fragment that contains EPO-cDNA and the 5′ end of CTP. Primer 1 contains the 5′ end of EPO cDNA sequence, which includes a new Age I site. Primer 3 contains the first four codons of the CTP and a stretch of the 3′ of EPO-cDNA. In the second reaction, pM² hCGβ primers 2 and 4 were used to synthesize a product containing the 3′ end of EPO-cDNA and the CTP sequence. Primer 4 contains the 3′ end of hCGβ sequence, which includes a new BamHI site. Primer 2 contains a stretch of the 3′ of EPO-cDNA and the first four codons of the CTP. In the third reaction, the two fragments obtained in reactions 1 and 2 were used as overlapping templates for an additional PCR step with primers 1 and 4. The resulting construct contains fused EPO-cDNA and CTP sequence.

The PCR generated construct was completely sequenced to ensure that no errors were introduced during the PCR. The AgeI/BamHI fragment containing the EPO-CDNA-CTP gene was inserted at the AgeI/BamHI cloning site of the eukaryotic expression vector, pTG123 (Invitrogen, San Diego, Calif.).

The pTG-EPO-CTP plasmid was transfected into CHO cells and stable clones were selected by adding zeocin antibiotics. The EPO-CTP protein is efficiently secreted from CHO cells into the medium as detected by Western blotting.

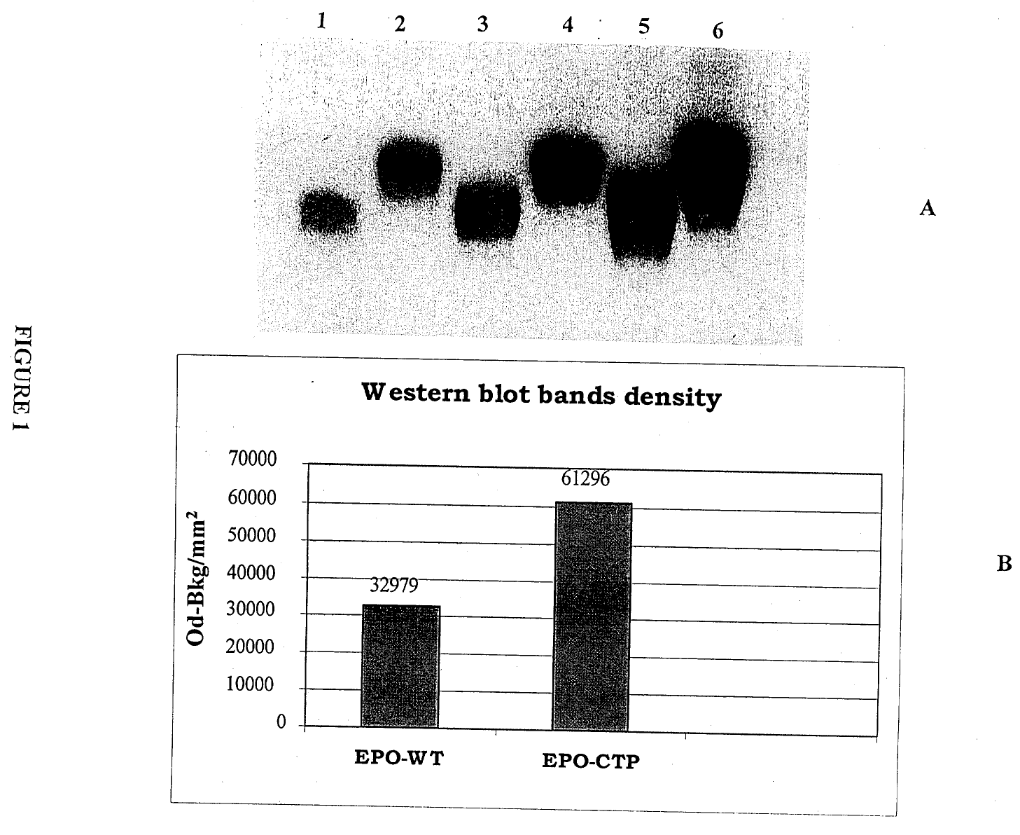

Surprisingly, the EPO-CTP protein is much more efficiently secreted from CHO cells than is wild type erythropoietin by a factor of approximately 1.85. These results are shown in FIG. 1 from an illustrative culture.

FIG. 1A shows the level of secretion at increasing times from the culture; lanes 1, 3 and 5 represent the wild type EPO secretion levels and lanes 2, 4 and 6, represent secretion at comparable time of EPO-CTP. Thus, in addition to providing an extended half-life, the addition of CTP onto the erythropoietin amino acid sequence results in an increased efficiency of production.

FIG. 1B is a graphical representation of cumulative secretion as shown in FIG. 1A.

EPO-CTP binds to EPO receptor with high affinity, because CTP is ligated to EPO in a region that not important for receptor binding and biological activity. Furthermore, it has a longer half-life in vivo and higher biological activity than wild type EPO.

1. A human form of erythropoietin extended at its C-terminus by the carboxy terminal peptide derived from the β subunit of human chorionic gonadotropin, which extended protein is recombinantly produced and secreted from Chinese hamster ovary cells.

2. A pharmaceutical composition which comprises the extended erythropoietin of claim 1.

3. A method to enhance red blood cell production which method comprises administering to a subject in need of said red blood cell proliferation an effective amount of the pharmaceutical composition of claim 2.
